(19) United States
(12) Patent Application Publication
(10) Pub. No.: US 2022/0364270 A1
(43) Pub. Date: Nov. 17, 2022

(54) STRETCHABLE NANO-MESH BIOELECTRODE AND METHOD OF FABRICATING THE SAME

(71) Applicant: DAEGU GYEONGBUK INSTITUTE OF SCIENCE AND TECHNOLOGY, Daegu (KR)

(72) Inventors: Sungwon LEE, Dangjin-si (KR); Wooseong JEONG, Daegu (KR); Seungsun YOO, Anseong-si (KR); Seon Min LEE, Namyangju-si (KR)

(21) Appl. No.: 17/525,590

(22) Filed: Nov. 12, 2021

(30) Foreign Application Priority Data
May 11, 2021 (KR) 10-2021-0060444

(51) Int. Cl. D01D 5/00; A61B 5/25; D01D 7/00; D06B 1/02

(52) U.S. Cl. CPC D01D 5/0038; A61B 5/25; D01D 7/00; D06B 1/02; A61B 2562/125; B82Y 5/00

(57) ABSTRACT

The present invention relates to a stretchable nano-mesh bioelectrode having excellent air permeability and durability. Specifically, the stretchable nano-mesh bioelectrode includes a nanofiber elastic mesh sheet including polymer nanofibers formed by electrospinning; and a metal nanowire network having a portion impregnated onto the nanofiber elastic mesh sheet.

### CROSS-REFERENCE TO RELATED APPLICATIONS

This application is based on and claims priority under 35 U.S.C. § 119 to Korean Patent Application No. 10-2021-0060444, filed on May 11, 2021, in the Korean Intellectual Property Office, the disclosure of which is herein incorporated by reference in its entirety.

### TECHNICAL FIELD

The present invention relates to a stretchable nano-mesh bioelectrode and a method of fabricating the same, and more particularly, to a stretchable nano-mesh bioelectrode having excellent air permeability, flexibility, and durability, and a method of fabricating the same.

### BACKGROUND ART

A bioelectrode is a device designed to transmit and receive electrical signals to/from organs and tissues of the body, and thus is used for the purpose of being implanted into the human body and/or attached to the epidermis of the skin to electrically interact with tissues and cells.

Specifically, a bioelectrode comes into contact with a certain part of the body to record electrical signals emitted from the body for a long or short period of time or deliver electrical stimuli to the body, thereby regulating the electrical activity of the cells and tissues and conducting research on various diseases using an electrical therapy.

A bioelectrode has been used for implantation into the heart, muscle, brain tissues, and the like in which the physiological state of the body is indicated by electrical signals, or has been used for attachment to the epidermis to monitor vital signals. For an elaborated interaction in biological environments, the bioelectrode requires durability to various changes such as low impedance capable of mediating fine electrical signals in vivo, a stable interaction with biological tissues, excellent biocompatibility, stretching, releasing, twisting, and bending of electrodes, and the like. Accordingly, research on development of a bioelectrode material has been actively conducted to satisfy the requirements.

To satisfy these requirements, Korean Registered Patent Publication No. 10-1284373 discloses a conductive polydimethylsiloxane composite composition containing a conductive filler having an aspect ratio of 1 or more, which may be used as a skin electrode.

However, when the electrode is formed based on the polydimethylsiloxane, it is difficult to form a metal layer due to the heterogeneity in terms of materials, such as a difference in lattice constants and thermal expansion coefficients between the metal layer and the polydimethylsiloxane as a silicone-based organic polymer. Also, because the above-described electrode has a weak adhesive force between the metal layer and the polydimethylsiloxane, the electrode has a problem in that, when the metal layer is patterned in a line width with units of micrometers, the metal layer and the polydimethylsiloxane may be easily separated from each other by modification of the electrode. Further, the electrode has a drawback in that it is difficult to attach it to the skin to monitor the vital signals for a long time due to the excretion of sweat produced in the human body, or the degraded gas permeability when the electrode is attached to the skin.

Accordingly, there is a need for development of a bioelectrode having excellent electrical characteristics and improved air permeability and durability to its modification so that it can be implanted into and/or attached to the human body for a long time to stably monitor the vital signals.

### RELATED ART DOCUMENT

### Patent Document

Korean Registered Patent Publication No. 10-1284373

### DISCLOSURE

### Technical Problem

An object of the present invention is to provide a stretchable nano-mesh bioelectrode having excellent air permeability, flexibility, and electrical characteristics.

Another object of the present invention is to provide a stretchable nano-mesh bioelectrode having significantly improved durability.

Still another object of the present invention is to provide a method of fabricating a stretchable nano-mesh bioelectrode, which is economical and with which it is easy to fabricate the bioelectrode.

### Technical Solution

In one general aspect, a stretchable nano-mesh bioelectrode includes a nanofiber elastic mesh sheet including polymer nanofibers formed by electrospinning; and a metal nanowire network having a portion impregnated onto the nanofiber elastic mesh sheet.

In the stretchable nano-mesh bioelectrode according to one embodiment of the present invention, a contact point between metal nanowires in the metal nanowire network may include a welding point.

In the stretchable nano-mesh bioelectrode according to one embodiment of the present invention, the impregnation onto the nanofiber elastic mesh sheet may be performed so that 20% by volume or more of the metal nanowire network is impregnated in a thickness direction.

In the stretchable nano-mesh bioelectrode according to one embodiment of the present invention, the nano-mesh bioelectrode may include pores, and the pores may be formed by the nanofiber mesh sheet onto which the metal nanowire network is impregnated.

In the stretchable nano-mesh bioelectrode according to one embodiment of the present invention, the polymer may include one or more selected from an olefin-based elastomer, a styrene-based elastomer, a thermoplastic polyester-based elastomer, a thermoplastic polyurethane-based elastomer, a thermoplastic acrylic elastomer, a thermoplastic vinyl-based polymer, a thermoplastic fluorine-based polymer, and a mixture thereof.

In the stretchable nano-mesh bioelectrode according to one embodiment of the present invention, the polymer may have a glass transition temperature of 60° C. or lower.

In the stretchable nano-mesh bioelectrode according to one embodiment of the present invention, a diameter ratio of the metal nanowires and the polymer nanofiber may be in a range of 1:5 to 1:100.

In the stretchable nano-mesh bioelectrode according to one embodiment of the present invention, the metal nanowires may have a diameter of 1 to 80 nm.

In the stretchable nano-mesh bioelectrode according to one embodiment of the present invention, the metal nanowires may have an aspect ratio of 100 to 1,500.

In the stretchable nano-mesh bioelectrode according to one embodiment of the present invention, when a stretching-releasing cycle is performed 500 times by applying a strain of 20% to the nano-mesh bioelectrode, a change in resistance of the nano-mesh bioelectrode may be less than or equal to 5 folds of an initial resistance value before the application of the strain.

In another general aspect, a strain sensor includes the stretchable nano-mesh bioelectrode provided according to one aspect of the present invention.

In still another general aspect, a method of fabricating the stretchable nano-mesh bioelectrode according to one aspect of the present invention is provided.

In yet another general aspect, a method of fabricating a stretchable nano-mesh bioelectrode includes: a) fabricating a nanofiber elastic mesh sheet including polymer nanofibers on a substrate using electrospinning; b) injecting a metal nanowire ink including a dispersion medium onto the nanofiber elastic mesh sheet fabricated on the substrate in the form of droplets using a spray injection method to coat the nanofiber elastic mesh sheet with metal nanowires; c) sintering the coated metal nanowires with light to fabricate a metal nanowire network in which some of the metal nanowires are impregnated onto the nanofiber elastic mesh sheet; and d) removing the substrate.

In the method of fabricating a stretchable nano-mesh bioelectrode according to one embodiment of the present invention, in step b), the metal nanowire ink may be injected in the form of droplets in a state in which the substrate is warmed at a temperature of 40 to 130° C.

In the method of fabricating a stretchable nano-mesh bioelectrode according to one embodiment of the present invention, the light sintering may be performed by irradiation with intense pulsed light (IPL).

In the method of fabricating a stretchable nano-mesh bioelectrode according to one embodiment of the present invention, the metal nanowires may be irradiated with the light at a light energy of 0.01 to 10 J/cm² for 0.1 to 10 milliseconds (ms).

In the method of fabricating a stretchable nano-mesh bioelectrode according to one embodiment of the present invention, a contact area between the metal nanowires may be welded by the light sintering in step c) to form a metal nanowire network.

### Advantageous Effects

The stretchable nano-mesh bioelectrode according to the present invention can have excellent air permeability and flexibility because the stretchable nano-mesh bioelectrode has a nano-mesh structure. Also, when a portion of the metal nanowire network is impregnated onto the nanofiber elastic mesh sheet, the stretchable nano-mesh bioelectrode according to the present invention can have significantly improved durability because the electrical characteristics of the electrode are maintained even when the electrode is modified.

Also, in the stretchable nano-mesh bioelectrode of the present invention, the metal nanowire network can include a welding point between the metal nanowires, and thus can have excellent electrical characteristics by reducing contact resistance.

Further, the electrical characteristics and durability of the stretchable nano-mesh bioelectrode according to the present invention can be improved by the light sintering. Therefore, a process of fabricating the stretchable nano-mesh bioelectrode has an advantage in that the incurred cost and process time may be saved because the process is easy to perform.

### DESCRIPTION OF DRAWINGS

FIG. 1 is a schematic diagram schematically showing a process of fabricating a stretchable nano-mesh bioelectrode.

FIGS. 2A and 2D are digital images of Example 1 and Comparative Example 1, respectively, FIGS. 2B and 2C are SEM images of Example 1, and FIGS. 2E and 2F are SEM images of Comparative Example 1.

FIG. 3 is a diagram showing sheet resistance characteristics of Examples 1 to 3 and Comparative Examples 1 to 3.

FIG. 4A shows digital images of Example 1 in which stretchable nano-mesh bioelectrode is stretched by 0%, 10%, 20%, 30%, 40%, and 50%, respectively, and FIG. 4B, FIG. 4C, FIG. 4D, FIG. 4E, and FIG. 4F are diagrams showing the results showing the trends of changes in resistance of Example 1 and Comparative Example 1 when a stretching-releasing cycle is performed 500 times with respect to 10%, 20%, 30%, 40%, and 50% strains, respectively.

FIG. 5A is a diagram showing a change in resistance according to a degree of strain of a strain sensor to which Example 1 is applied, FIG. 5B is a digital image showing situations when a mouse is clicked and when a mouse is not clicked after the strain sensor is attached to a finger, and FIG. 5C is a diagram showing the results of observing a change in resistance in a real-time manner when the mouse is clicked and when the mouse is not clicked in a state in which the strain sensor is attached to a finger.

### BEST MODE

Hereinafter, the present invention will be described in further detail with reference to embodiments or examples thereof in conjunction with the accompanying drawings. However, it should be understood that the following embodiments or examples are illustrative only to describe the present invention in detail, but are not intended to limit the scope of the present invention, and may be embodied in various forms.

Also, unless otherwise defined, all technical and scientific terms used herein have the same meaning as commonly understood by one of ordinary skill in the art to which the present invention pertains. The terms used in the description in the present invention are given only for effectively describing specific embodiments and are not intended to limit the present invention.

Also, the singular forms used in the specification and the appended claims may be intended to include the plural forms as well, unless the context clearly indicates otherwise.

Further, a certain part “including” a certain element signifies that the certain part may be further inclusive, instead of exclusive, of another element unless particularly indicated otherwise.

A stretchable nano-mesh bioelectrode according to an aspect of the present invention includes a nanofiber elastic mesh sheet including polymer nanofibers formed by electrospinning; and a metal nanowire network having a portion impregnated onto the nanofiber elastic mesh sheet.

Specifically, because the nanofiber elastic mesh sheet included in the stretchable nano-mesh bioelectrode of the present invention is formed by electrospinning, the nanofiber elastic mesh has excellent air permeability and flexibility. Therefore, the stretchable nano-mesh bioelectrode may have advantages in that moisture or sweat is easily excreted when the bioelectrode is implanted into and/or attached to the body, and the vital signals may be measured or monitored for a long time because harmful gases that may be produced in the human body are easily discharged. Also, the stretchable nano-mesh bioelectrode has advantages in that it is easily fixed in the skin or tissue in vivo, and is not easily detached from the tissue because it is flexibly bent and elongated even with the movement of the human body.

Also, because the metal nanowire network included in the stretchable nano-mesh bioelectrode of the present invention has a structure having a portion impregnated onto the nanofiber elastic mesh sheet, the metal nanowires are not easily removed from a surface of the nanofiber elastic mesh sheet due to a strong physical bond between the metal nanowires and the nanofiber elastic mesh sheet. Therefore, a change in resistance may be reversible according to various modifications (such as stretching, releasing, twisting, bending, and the like) of the bioelectrode, and the bioelectrode is free from the problems regarding the detachment of the metal nanowire network when the bioelectrode is modified and then returns to its original state. Therefore, the stretchable nano-mesh bioelectrode may have excellent durability because the initial electrical characteristics of the bioelectrode may be maintained.

As one example, the stretchable nano-mesh bioelectrode of the present invention may be applicable to a skin-attachable or in vivo implantable bioelectrode.

Specifically, the skin-attachable stretchable nano-mesh bioelectrode may be used to check vital signal using an electrocardiograph (ECG), an electromyogram (EMG), an electroencephalogram (EEG), or the like. The in vivo implantable stretchable nano-mesh bioelectrode may be used to apply stimuli to abnormal tissues such as nerve system tissues, tumors, or the like.

Hereinafter, respective configurations of the stretchable nano-mesh bioelectrode according to an aspect of the present invention will be described in detail.

The stretchable nano-mesh bioelectrode of the present invention may include a metal nanowire network having a portion impregnated onto the nanofiber elastic mesh sheet.

The metal nanowire network may have a contact point at which the metal nanowires are crossed to come into contact with each other, and may also have a network structure.

According to one embodiment, the contact point between the metal nanowires in the metal nanowire network may include a welding point.

Specifically, because the metal nanowire network includes a welding point, the stretchable nano-mesh bioelectrode may have excellent electrical characteristics due to the significantly reduce contact resistance, thereby significantly improving the durability of the bioelectrode.

The conventional bioelectrodes have drawbacks in that they have high contact resistance because the metal nanowires are electrically connected by the physical contact between conductive fillers having an aspect ratio of 1 or more, and the bioelectrodes may be degraded due to a local increase in temperature at a contact point caused by the high contact resistance, resulting in degraded durability of the bioelectrodes. Also, a change in resistance of the bioelectrode with respect to the initial resistance may be caused due to a change in a contact area or a degree of contact when the bioelectrode is modified and then returns to its original state. When this process is repeated, the electrical characteristics of the bioelectrode may be eventually highly degraded.

On the other hand, because the stretchable nano-mesh bioelectrode of the present invention includes a metal nanowire network in which the contact point between the metal nanowires includes a welding point, the contact resistance may be significantly degraded by a welding rather than the simple physical contact, and the welding point may be maintained when the bioelectrode is modified and then returns to its original state. Therefore, the initial electrical characteristics of the bioelectrode may be maintained, thereby significantly improving the durability of the bioelectrode, compared to the prior art.

According to one embodiment of the present invention, the metal nanowire network may have a portion impregnated onto the nanofiber elastic mesh sheet.

According to one specific embodiment, 20% by volume or more, 30% by volume or more, 40% by volume or more, 50% by volume or more, and 90% by volume or less of the metal nanowire network may be impregnated onto the nanofiber elastic mesh sheet in a thickness direction.

When a portion of the metal nanowire network is impregnated onto the nanofiber elastic mesh sheet in a thickness direction, a strong physical bond between the metal nanowires and the nanofiber elastic mesh sheet may be formed. As a result, because the metal nanowires have an advantage in that they are not easily removed from a surface of the nanofiber elastic mesh sheet, detachment of the metal nanowires from the nanofiber elastic mesh sheet, which may be caused by various modifications such as stretching, releasing, twisting, bending, and the like, may be effectively prevented, thereby significantly improving the durability of the bioelectrode.

However, when less than 20% by volume of the metal nanowire network is impregnated onto the nanofiber elastic mesh sheet in a thickness direction, a portion of the metal nanowire network may be peeled from the nanofiber elastic mesh sheet due to the modification of the bioelectrode. When more than 90% by volume of the metal nanowire network is impregnated onto the nanofiber elastic mesh sheet, thermal modification of the nanofiber elastic mesh sheet may be caused in the case of a method of fabricating a stretchable nano-mesh bioelectrode as will be described below. Therefore, it is desirable that the percentage by volume (% by volume) of the metal nanowire network, which falls within the above range, is impregnated onto the nanofiber elastic mesh sheet in a thickness direction in order to efficiently improve the durability of the bioelectrode.

As an example, a metal having excellent conductivity and showing biocompatibility without causing a rejection reaction or damage to the human body as well is used as the metal nanowires. Specific examples of the metal nanowires may include one or more selected from gold nanowires, platinum nanowires, and silver nanowires.

As another example, the metal nanowires may be core-shell nanowires coated with the above-described biocompatible metal. The core material may be selected from copper, nickel, carbon black, zinc, aluminum, graphite, graphene, and the like, but the present invention is not limited thereto.

According to one specific embodiment, the metal nanowires may have a diameter of 1 to 80 nm, specifically a diameter of 10 to 70 nm, and more specifically a diameter of 20 to 70 nm.

According to another specific embodiment, the metal nanowires may have an aspect ratio of 100 to 1,500, desirably an aspect ratio of 300 to 1,200, more desirably an aspect ratio of 500 to 1,000.

It is desirable that the metal nanowires have the diameter and aspect ratio falling within the above ranges in order to sufficiently secure the welding point between the metal nanowires as described above and smoothly impregnate the metal nanowire network onto the nanofiber elastic mesh sheet.

According to one embodiment, the nanofiber elastic mesh sheet included in the stretchable nano-mesh bioelectrode of the present invention may have a structure in which polymer nanofiber strands formed by electrospinning are intermingled in the form of a network.

When the nanofiber elastic mesh sheet having a structure in which the polymer nanofiber strands are intermingled in the form of a network is included in the stretchable nano-mesh bioelectrode of the present invention, it is possible to secure the excellent air permeability. Therefore, the stretchable nano-mesh bioelectrode of the present invention has an advantage in that moisture, sweat, or gases produced in the human body may be easily excreted when the bioelectrode of the present invention is implanted into and/or attached to the body.

Also, because flexibility may be secured by the structure of the nanofiber elastic mesh sheet in which the polymer nanofiber strands are intermingled in the form of a network, the bioelectrode may be fixed in the skin or tissue in vivo, which may be desirable. Also, the bioelectrode has an advantage in that it is not easily detached from the tissue because it is flexibly bent and elongated even with the movement of the human body. Therefore, it is possible to measure or monitor the vital signals for a long time using the stretchable nano-mesh bioelectrode including the nanofiber elastic mesh sheet.

According to one embodiment, the nanofiber elastic mesh sheet may have a thickness of 700 nm to 10 μm, desirably a thickness of 800 nm to 5 μm, and more desirably a thickness of 1 to 3 μm. The stretchable nano-mesh bioelectrode may have an excellent degree of flexibility similar to the skin within this range, and may be easily attached to the skin or tissue in vivo.

Also, in the case of the method of fabricating a stretchable nano-mesh bioelectrode as described below, it is desirable that the thickness of the nanofiber elastic mesh sheet satisfies the above range in order to suppress the modification of the nanofiber elastic mesh sheet that may be caused by heat.

The nanofiber elastic mesh sheet includes polymer nanofibers formed by electrospinning. In this case, the formation of the polymer nanofibers by electrospinning will be described in further detail in the method of fabricating a stretchable nano-mesh bioelectrode, which is another aspect of the present invention.

In this case, a polymer included in the polymer nanofibers formed by electrospinning may be used without any limitation as long as it can be used in the human body. As one example, the polymer may include one or more selected from an olefin-based elastomer, a styrene-based elastomer, a thermoplastic polyester-based elastomer, a thermoplastic polyurethane-based elastomer, a thermoplastic acrylic elastomer, a thermoplastic vinyl-based polymer, a thermoplastic fluorine-based polymer, and a mixture thereof. In this case, it goes without saying that the polymer may be selected according to a purpose of the bioelectrode, for attachment to the skin or implantation into the living body.

As a specific example, the polymer may include one or a mixture of two or more selected from polyvinyl alcohol (PVA), polyethylene glycol (PEG), polypropylene glycol (PPG), polyacrylic acid (PAA), carboxymethyl cellulose (CMC), polyvinylpyrrolidone (PVP), thermoplastic polyurethane (TPU), and polyvinylidene fluoride-trifluoroethylene (P(VDF-TrFE)).

As an example, the polymer may be used without any particular limitation as long as the polymer may have a suitable molecular weight to form the polymer nanofibers by electrospinning. Specifically, the polymer may have a weight average molecular weight of 10,000 to 500,000 g/mol, desirably a weight average molecular weight of 30,000 to 300,000 g/mol, more desirably a weight average molecular weight of 50,000 to 200,000 g/mol, and most desirably a weight average molecular weight of 80,000 to 150,000 g/mol.

According to one example of the present invention, the polymer may have a glass transition temperature of 60° C. or lower. Specifically, the polymer may have a glass transition temperature of 40° C. or lower, more specifically 25° C. or lower. In this case, the lower limit of the glass transition temperature is not particularly limited, and may be −100° C.

When the glass transition temperature of the polymer satisfies the above range, it may be favorable for fabricating a metal nanowire network in which some of the metal nanowires are impregnated onto the nanofiber elastic mesh sheet including the polymer nanofibers in the method of fabricating a stretchable nano-mesh bioelectrode as described below.

According to one embodiment, a diameter ratio of the metal nanowires and the polymer nanofiber is in a range of 1:5 to 1:100, desirably 1:10 to 80, and more desirably 1:20 to 60.

As previously described above, when the nanofiber elastic mesh sheet in which the polymer nanofibers are intermingled in the form of a network may retain air permeability and flexibility, and some of the metal nanowires are impregnated onto the nanofiber elastic mesh sheet to construct a metal nanowire network, a stretchable nano-mesh bioelectrode having excellent durability and electrical characteristics may be provided. In this case, it is favorable to impregnate the metal nanowires onto the nanofiber elastic mesh sheet. After the impregnation of the metal nanowires, that is, in order for the stretchable nano-mesh bioelectrode to maintain the air permeability and flexibility attributed from the nanofiber elastic mesh sheet, the diameter ratio of the metal nanowires and the polymer nanofiber satisfies the above range, which is desirable.

According to one specific embodiment, the nano-mesh bioelectrode includes pores, and the pores may be formed by the nanofiber mesh sheet onto which the metal nanowire network is impregnated.

When the nano-mesh bioelectrode includes pores, the sweat or gases produced in the human body may be easily excreted, which makes it possible to measure or monitor the vital signals for a long time. Such pores may be primarily provided from the nanofiber elastic mesh sheet in which the above-described polymer nanofibers are intermingled in the form of a network. Also, when the diameter ratio of the metal nanowires and the polymer nanofiber satisfies the above range, the pores may be provided even when the bioelectrode includes the metal nanowire network in which the metal nanowires are impregnated onto the nanofiber elastic mesh sheet. That is, because the pores included in the nano-mesh bioelectrode may be formed by the nanofiber mesh sheet onto which the metal nanowire network is impregnated, the bioelectrode that may be used for a long time may be provided.

As one example, the pores may have a size of 1 nm to 100 μm, specifically a size of 50 nm to 50 μm, and more specifically a size of 100 nm to 10 μm.

As previously described above, the stretchable nano-mesh bioelectrode provided according to an aspect of the present invention may be used for a long time because the stretchable nano-mesh bioelectrode has excellent air permeability and flexibility. When the structure in which a portion of the metal nanowire network in which the contact point between the metal nanowires includes a welding point is impregnated on the nanofiber elastic mesh sheet is provided, the stretchable nano-mesh bioelectrode may have excellent sheet resistance characteristics and significantly improved durability as well.

According to one specific embodiment, the nano-mesh bioelectrode may have a sheet resistance of 1 to 30 Ω/sq, specifically a sheet resistance of 1 to 20 Ω/sq, and more specifically a sheet resistance of 2 to 10 Ω/sq.

This means that the bioelectrode of the present invention may have low sheet resistance characteristics falling within the above range because the contact resistance of the metal nanowire network is significantly reduced when the contact points between the metal nanowires are connected by welding rather than the simple physical contact.

According to one embodiment, when a stretching-releasing cycle is performed 500 times by applying a strain of 20% to the nano-mesh bioelectrode, a change in resistance of the nano-mesh bioelectrode may be less than or equal to 5 folds, desirably less than or equal to 3 folds, and more desirably less than or equal to 2 folds of an initial resistance value before the application of the strain. In this case, the lower limit of the change in resistance may be greater than or equal to 1.1 folds.

Specifically, the stretchable nano-mesh bioelectrode of the present invention has a structure of the metal nanowire network including a welding point and a structure in which a portion of the metal nanowire network is impregnated onto the nanofiber elastic mesh sheet. Therefore, when the bioelectrode is stretched and then returns to its original state, the resistance value of the bioelectrode is maintained similarly with the initial resistance value of the bioelectrode before the bioelectrode is modified. As such, it can be seen that the bioelectrode of the present invention has excellent durability to stably measure or monitor the vital signals because the bioelectrode is free from the problem regarding the detachment of the metal nanowire network even when the bioelectrode is attached to a body part (for example, a joint area) that may be modified.

According to still another aspect of the present invention, a strain sensor including the stretchable nano-mesh bioelectrode of the present invention may be provided.

As an example, the stretchable nano-mesh bioelectrode of the present invention may be easily modified due to the excellent flexibility, and the sheet resistance of the stretchable nano-mesh bioelectrode may be reversibly changed according to the applied strain. Also, when the bioelectrode returns to its original state, there is no change in resistance of the bioelectrode relative to the initial resistance value. Therefore, the stretchable nano-mesh bioelectrode may be applied to a strain sensor having excellent reliability and durability.

The type of strain causing a change in sheet resistance is not particularly limited as long as the sheet resistance of the strain sensor is reversibly varied according to the modification, and the strain sensor is modified and then returns to its original state. As an example, the type of strain may include one or more selected from stretching, releasing, twisting, and bending. That is, the strain sensor may detect a fine mechanical change based on a change in resistance sensed according to a degree of strain applied to the strain sensor including the stretchable nano-mesh bioelectrode.

According to yet another aspect of the present invention, a method of fabricating a stretchable nano-mesh bioelectrode is provided.

The method of fabricating a stretchable nano-mesh bioelectrode according to an embodiment of the present invention includes: a) fabricating a nanofiber elastic mesh sheet including polymer nanofibers on a substrate using electrospinning; b) injecting a metal nanowire ink including a solvent onto the nanofiber elastic mesh sheet fabricated on the substrate in the form of droplets using a spray injection method to coat the nanofiber elastic mesh sheet with metal nanowires; c) sintering the metal nanowires with light to fabricate a metal nanowire network in which some of the metal nanowires are impregnated onto the nanofiber elastic mesh sheet; and d) removing the substrate.

After the nanofiber elastic mesh sheet including the polymer nanofibers is first formed by electrospinning in this way, the nanofiber elastic mesh sheet is coated with the metal nanowires using a spray injection method, and the metal nanowires are then sintered with light to fabricate a metal nanowire network in which some of the metal nanowires are impregnated onto the nanofiber elastic mesh sheet, thereby fabricating a stretchable nano-mesh bioelectrode. Therefore, the stretchable nano-mesh bioelectrode has an advantage in that a microelectrode is relatively easily fabricated by an economical method because it is not difficult to finely process the bioelectrode. Also, because the structural stability is improved when a portion of the metal nanowire network is impregnated onto the nanofiber elastic mesh sheet using a simple process such as light sintering, the bioelectrode having excellent durability may be provided, compared to the conventional bioelectrodes.

Hereinafter, respective steps of the method of fabricating a stretchable nano-mesh bioelectrode will be described in further detail. However, the types of respective building materials and the like are as described for the stretchable nano-mesh bioelectrode, and thus a description of the same materials will be omitted.

First, step a) of fabricating a nanofiber elastic mesh sheet including polymer nanofibers on a substrate using electrospinning may be performed.

As an example, the electrospinning may be performed using a method commonly used in the related art. Specifically, when a syringe is filled with a polymer solution and a high voltage is applied to the polymer solution while discharging the polymer solution through a needle tip, the polymer solution in a liquid phase may be produced into nanofibers in a collector using an electric field generated at the high voltage.

More specifically, the polymer solution according to an embodiment of the present invention is fabricated by dissolving the above-described polymer in a solvent. In the polymer solution, the polymer may be included at 5 to 30% by weight, desirably 8 to 20% by weight, and most desirably 10 to 15% by weight. When the polymer in the polymer solution is included within the above range, the nanofibers formed by electrospinning may form continuous-phase fibers without being cut into various filaments, which may be desirable.

In this case, the solvent may include one or more selected from distilled (DI) water, acetone, ethanol, N,N-dimethylene acetamide (DMAc), N-methyl pyrrolidone (NMP), dimethyl sulfoxide (DMSO), methyl ethyl ketone (MEK), and N,N-dimethylformamide (DMF).

In addition, a separation distance between the needle tip and the collector, a strength of voltage, and a discharge rate of the polymer solution should be properly adjusted in order to effectively fabricate the nanofiber elastic mesh sheet.

The separation distance between the needle tip and the collector according to an embodiment of the present may be in a range of 5 to 50 cm, desirably 10 to 40 cm, and most desirably in a range of 15 to 30 cm. In this case, when the separation distance between the needle tip and the collector is too close, adhesion between the nanofibers may severely occur. On the other hand, when the separation distance is too remote, it is difficult to form continuous-phase fibers due to the evaporation of the solvent. Therefore, it is desirable that the separation distance between the needle tip and the collector satisfies the above range.

The strength of voltage according to an embodiment of the present invention is not particularly limited as long as it is a typical voltage strength applied to form the polymer nanofibers by electrospinning. Specifically, the strength of voltage may be, for example, in a range of 1 to kV, desirably in a range of 5 to 25 kV, and more desirably in a range of 10 to 20 kV. The electrospinning may be effectively performed within the above range.

The discharge rate according to an embodiment of the present invention is used to adjust a thickness of the polymer nanofibers as desired even without a break by adjusting an amount of the polymer solution to be discharged. Specifically, the discharge rate of the polymer solution may be, for example, in a range of 0.1 to 20 mL/hr, more desirably in a range of 0.5 to 15 mL/hr, and most desirably in a range of 0.8 to 10 mL/hr. Within such a range, the polymer nanofibers having a thickness as desired without a break may be easily fabricated.

Next, the method of fabricating a stretchable nano-mesh bioelectrode may include step b) of injecting a metal nanowire ink including a dispersion medium onto the nanofiber elastic mesh sheet fabricated on the substrate in the form of droplets using a spray injection method to coat the nanofiber elastic mesh sheet with metal nanowires.

Specifically, the coating with the metal nanowires may be performed by exposing only a portion of the metal nanowires to be coated onto the nanofiber elastic mesh sheet to coat a portion or all of the nanofiber elastic mesh sheet with the metal nanowires as desired. As a more specific example, a designed shadow mask is placed on the nanofiber elastic mesh sheet, and a region of the nanofiber elastic mesh sheet which is not covered by the shadow mask, that is, a region exposed to the outside, may be coated with the metal nanowires. In this case, as previously described above, the metal nanowires may include one or more selected from gold nanowires, platinum nanowires, and silver nanowires. Also, the metal nanowires may be core-shell nanowires coated with a biocompatible metal.

According to one embodiment of the present invention, the coating with the metal nanowires may be performed by injecting a metal nanowire ink including a dispersion medium in the form of droplets using a spray injection method to coat the nanofiber elastic mesh sheet with the metal nanowires.

When the metal nanowire ink is injected in the form of droplets using a spray injection method, the metal nanowire ink may be uniformly injected onto the nanofiber elastic mesh sheet, and an aggregation between the metal nanowires may be suppressed, which makes it desirable to fabricate a metal nanowire network as described below.

In this case, materials known in the related art for the purpose of suppressing the aggregation between the metal nanowires and simultaneously enhancing dispersibility are used as the dispersion medium without any particular limitation. As one example, the dispersion medium may include one or more selected from methanol, ethanol, ethylene glycol, toluene, terpineol, acetonitrile, and isopropanol.

According to one specific embodiment, the metal nanowire ink may contain 0.01 to 10% by weight, desirably 0.05 to 5% by weight, and more desirably 0.1 to 1% by weight of the metal nanowires.

As previously described above, the metal nanowire may have a diameter of 1 to 80 nm, specifically a diameter of 10 to 70 nm, and more specifically a diameter of 20 to 70 nm, and the metal nanowire may have an aspect ratio of 100 to 1,500, desirably an aspect ratio of 300 to 1,200, and more desirably an aspect ratio of 500 to 1,000. Preferably, the metal nanowire ink contains the metal nanowires within the above range in order to disperse the metal nanowires having such morphological characteristics in the metal nanowire ink including a dispersion medium without any aggregation between the metal nanowires to uniformly inject the metal nanowires on the nanofiber elastic mesh sheet using a spray injection method.

According to one embodiment, the metal nanowire ink may be injected through a spray head in the form of droplets to coat the nanofiber elastic mesh sheet with the metal nanowires.

In this case, the spray head may be used to inject the metal nanowire ink at a distance spaced 1 to 50 cm, desirably 10 to 40 cm, and more desirably 20 to 40 cm apart from the nanofiber elastic mesh sheet. Also, the spray head may be used to inject the metal nanowire ink at an aslant angle of 0 to 90°, specifically 0 to 80°, and more specifically 0 to 45° in a left or right direction with respect to the gravity direction. It is desirable to perform a spray injection under the above-described condition because the nanofiber elastic mesh sheet may be uniformly coated with the metal nanowires to form a welding point at a contact point between the metal wires for fabrication of the metal nanowire network as will be described below and facilitate the impregnation of the metal nanowires onto the nanofiber elastic mesh sheet in a thickness direction.

According to one embodiment of the present invention, in step b) of coating the nanofiber elastic mesh sheet with the metal nanowires, the metal nanowire ink may be injected in the form of droplets in a state in which the substrate is warmed at a temperature of 40 to 130° C., specifically a temperature of 60 to 130° C., and more specifically a temperature of 80 to 130° C.

After the fabrication of the nanofiber elastic mesh sheet fabricated on the substrate, the metal nanowire ink may be injected onto the nanofiber elastic mesh sheet in the form of droplets in a state in which the substrate is warmed in the above temperature range. As a result, because the dispersion medium included in the droplets may be rapidly evaporated to effectively prevent the pores included in the above-described nanofiber elastic mesh sheet from being clogged with the droplets. Therefore, the bioelectrode of the present invention may have excellent air permeability and flexibility. When the dispersion medium is slowly evaporated, an aggregation between the metal nanowires included in the droplets easily occurs. Therefore, the pores included in the nanofiber elastic mesh sheet are clogged with the droplets, resulting in degraded air permeability and flexibility of the bioelectrode and degraded electrical characteristics due to the aggregation between the metal nanowires as well. Accordingly, it is very important to inject the metal nanowire ink in a state in which the substrate is warmed in the above temperature range.

In addition, when the substrate is warmed, the nanofiber elastic mesh sheet fabricated on the substrate may be indirectly heated, which makes it possible to prevent the modification of nanofiber elastic mesh sheet by heat. Also, this process finds an advantage of playing an advantageous role in impregnating the metal nanowire as described below into the nanofiber elastic mesh sheet in a thickness direction.

As an example, the substrate has a glass transition temperature higher than the polymer nanofiber included in the nanofiber elastic mesh sheet. In this case, a transparent substrate which does not absorb light energy by light sintering as described below is satisfied as the substrate, but the present invention is not limited thereto. Therefore, according to one specific embodiment, the substrate may be selected from polyethylene terephthalate (PET), polyethylene naphthalate (PEN), polyether ether ketone (PEEK), polycarbonate (PC), polyarylate (PAR), polyether sulfone (PES), or polyimide (PI), but the present invention is not limited thereto.

According to one embodiment of the present invention, the method of fabricating a stretchable nano-mesh bioelectrode may include step c) of sintering the metal nanowires with light to fabricate a metal nanowire network in which some of the metal nanowires are impregnated onto the nanofiber elastic mesh sheet.

According to one embodiment, the light sintering may be performed by irradiating the metal nanowires with intense pulsed light (IPL).

Specifically, the light sintering is performed by irradiating the metal nanowires with light having a desired wavelength in the form of pulses for a very short time to selectively transfer energy to the metal nanowires at a high speed. In this case, a surface temperature of the metal nanowires increases from 500 to 1,500° C. in a moment. Due to the temperature increased in a moment, some of the metal nanowires may be impregnated onto the nanofiber elastic mesh sheet. In this case, the impregnation of the metal nanowires may be efficiently achieved with the warming effect of the substrate as previously described above, as well as the instantly increased surface temperature of the metal nanowires.

According to one specific embodiment, 20% by volume or more, 30% by volume or more, 40% by volume or more, 50% by volume or more, and 90% by volume or less of the metal nanowire network may be impregnated onto the nanofiber elastic mesh sheet in a thickness direction.

Also, because photonic welding occurs at the contact point between the metal nanowires due to the increased surface temperature of the metal nanowires, the contact point may form a metal nanowire network including a welding point.

The bioelectrode of the present invention including the metal nanowire network including a welding point may have significantly reduced contact resistance, and thus may have excellent sheet resistance characteristics. Within the range of percentage by volume, because the metal nanowire network is impregnated onto the nanofiber elastic mesh sheet in a thickness direction, the welding point may be intactly maintained even when the bioelectrode of the present invention is modified and then returns to its original state. Also, because there is hardly a change in initial electrical characteristics of the bioelectrode, the durability of the bioelectrode according to the present invention may be significantly improved, compared to the conventional bioelectrodes.

As an example, the intense pulsed light may be light having a wavelength of 300 to 1,400 nm, specifically a wavelength of 500 to 1,200 nm, and more specifically a wavelength of 800 to 1,000 nm.

According to one specific embodiment, the energy transferred during the light sintering may be regulated by one or more factors selected from light intensity, a light irradiation time, a voltage, a pulse-to-pulse frequency, and a pulse number.

As an example, the light irradiated for light sintering has a light energy of 0.01 to 10 J/cm², specifically a light energy of 0.05 to 8 J/cm², and more specifically a light energy of 0.1 to 4 J/cm². In this case, the metal nanowires may be irradiated with light for 0.1 to 10 milliseconds (ms), desirably 0.5 to 8 ms, and more desirably 1 to 5 ms.

According to one embodiment, the metal nanowires may be irradiated with light by applying a voltage of 150 to 500 V, specifically a voltage of 200 to 400 V, and more specifically a voltage of 250 to 350 V. In this case, the pulse-to-pulse frequency may be in a range of 0.1 to 10 Hz, desirably in a range of 0.5 to 5 Hz, and more desirably in a range of 0.5 to 1.5 Hz, and the pulse number may be less than or equal to 10, specifically less than or equal to 5, and more specifically less than or equal to 3.

To form a welding point by photonic welding at the contact point between the metal nanowires as previously described above and efficiently impregnate the metal nanowire network onto the nanofiber elastic mesh sheet in a thickness direction while suppressing the thermal modification of the nanofiber elastic mesh sheet caused by the heat applied or accumulated during the light sintering, it is desirable to sinter the metal nanowires under the condition of the light sintering to fabricate the metal nanowire network.

The stretchable nano-mesh bioelectrode having excellent air permeability, flexibility, and durability according to the present invention may be fabricated by fabricating a metal nanowire network impregnated onto the nanofiber elastic mesh sheet using the above-described method, followed by removal of the substrate.

Hereinafter, the stretchable nano-mesh bioelectrode according to the present invention and the method of fabricating the same will be described in further detail with reference to embodiments thereof. However, it should be understood that the following embodiments are illustrative only to describe the present invention in detail, but are not intended to limit the scope of the present invention, and may be embodied in various forms.

Also, unless otherwise defined, all technical and scientific terms used herein have the same meaning as commonly understood by one of ordinary skill in the art to which the present invention pertains. The terms used in the description in the present invention are given only for effectively describing specific embodiments and are not intended to limit the present invention. In addition, the units of additives that are not described in this specification may be based on the percentage by weight.

### Example 1

Thermoplastic polyurethane (TPU; Serial Number: P22SRNAT, Miractran Co., Ltd.) was mixed in a solvent in which methyl ethyl ketone (MKE) and dimethylformamide (DMF) were mixed at a weight ratio of 5:5, and then stirred. Thereafter, a syringe was filled with 11.5% by weight of the TPU polymer solution, and the TPU polymer solution was then electrospun on a polyethylene terephthalate (PET) substrate using an electrospinning apparatus (ESR200R2, NanoNC, Korea). In this case, an inner diameter of a syringe needle was 0.31 mm, and a separation distance between the needle tip of the syringe and the PET substrate (a collector) was 20 cm. The TPU polymer solution was spun at a rate of 2 mL/hr for 5 minutes while applying a voltage of 15 kV to fabricate a nanofiber elastic mesh sheet in which nanofibers were intermingled in the form of a network.

Next, a designed shadow mask was placed on the nanofiber elastic mesh sheet, and a silver nanowire ink was injected onto some exposed region of the nanofiber elastic mesh sheet through this shadow mask using a spray injection method to coat the nanofiber elastic mesh sheet with silver nanowires. In this case, the silver nanowire ink was prepared by dispersing silver nanowires in ethanol so that the silver nanowires were included at 0.5% by weight, and the spray injection was performed by setting a spray head to be located in parallel with a gravity direction at a distance spaced 30 cm apart from the center of the nanofiber elastic mesh sheet, and applying a pneumatic pressure of 50 psi to inject the silver nanowire ink at a rate of 0.08 mL/s so that the nanofiber elastic mesh sheet was coated with 62 μg/cm² of the silver nanowires. The injection of the silver nanowire ink was performed after the substrate on which the nanofiber elastic mesh sheet was fabricated was placed on a 110° C. hot plate.

Then, the light sintering was performed under the conditions of a voltage of 300 V, a light irradiation time of 3 ms, a pulse-to-pulse frequency of 1 Hz, and a pulse number of 1 using intense pulsed light sintering equipment, and the PET substrate was then removed to fabricate a stretchable nano-mesh bioelectrode.

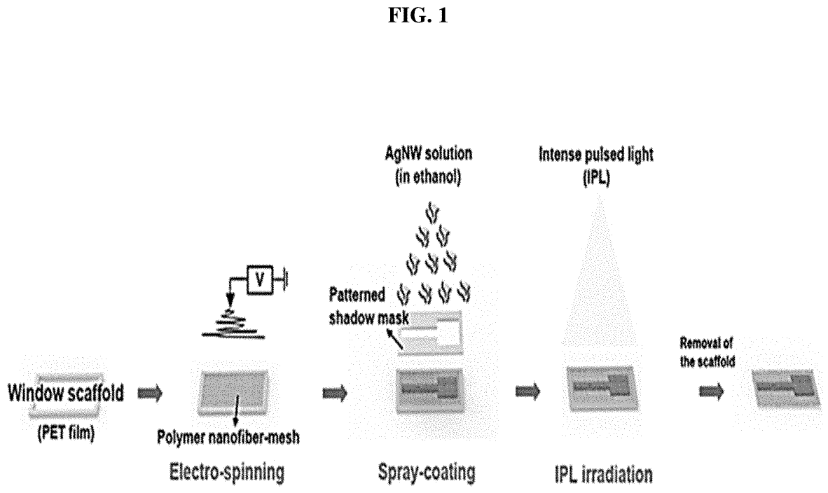

A process of fabricating a stretchable nano-mesh bioelectrode fabricated according to one embodiment of the present invention is schematically shown in FIG. 1.

### Example 2

A stretchable nano-mesh bioelectrode was fabricated in the same manner as in Example 1, except that a PVA polymer solution obtained by mixing polyvinyl alcohol (PVA; 98 to 99% hydrolyzed, M_{w}=130,000, Alfa Aesar) as the polymer in distilled water followed by stirring was electrospun.

### Example 3

A stretchable nano-mesh bioelectrode was fabricated in the same manner as in Example 1, except that a PVA polymer solution obtained by mixing polyvinylidene fluoride-trifluoroethylene (P(VDF-TrFE); FC 30, PIEZOTECH, Ltd.) as the polymer in acetone followed by stirring was electrospun.

### Comparative Example 1

A stretchable nano-mesh bioelectrode was fabricated in the same manner as in Example 1, except that a nanofiber elastic mesh sheet was coated with silver nanowires using a spray injection method, but a subsequent light sintering process was not performed.

### Comparative Example 2

A stretchable nano-mesh bioelectrode was fabricated in the same manner as in Example 2, except that a nanofiber elastic mesh sheet was coated with silver nanowires using a spray injection method, but a subsequent light sintering process was not performed.

### Comparative Example 3

A stretchable nano-mesh bioelectrode was fabricated in the same manner as in Example 3, except that a nanofiber elastic mesh sheet was coated with silver nanowires using a spray injection method, but a subsequent light sintering process was not performed.

### (Experimental Example 1) Comparison of Morphological Characteristics

The morphological characteristics of the bioelectrodes of Example 1 and Comparative Example 1 were analyzed using a digital camera and a scanning electron microscope (SEM) to compare the morphological characteristics before and after the light sintering.

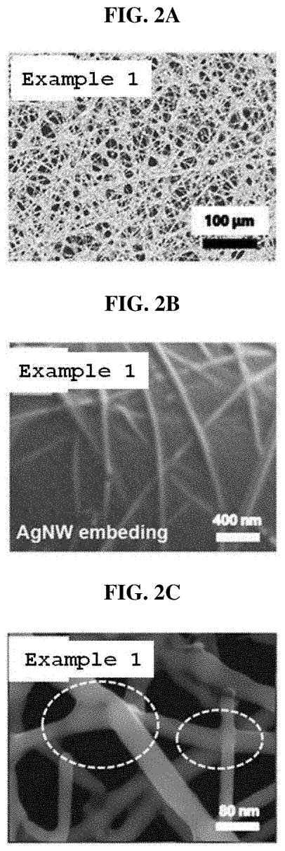

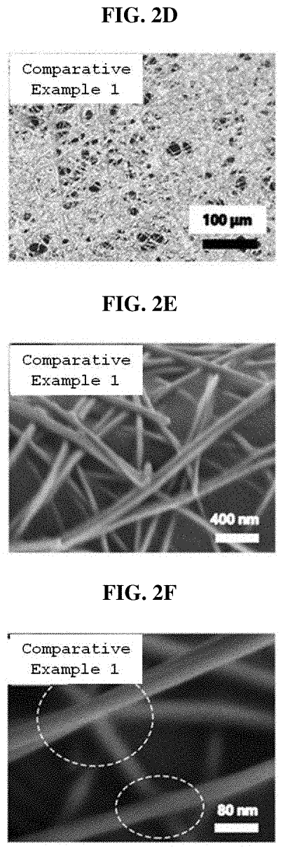

FIGS. 2A and 2D are diagrams showing digital images of Example 1 and Comparative Example 1, respectively, FIGS. 2B and 2C show SEM images of Example 1, and FIGS. 2E and 2F show SEM images of Comparative Example 1.

When FIGS. 2A and 2D were compared, it can be seen that a larger amount of pores was observed in Example 1, compared to Comparative Example 1. This indicates that some of the silver nanowires were impregnated onto the nanofibers in the case of Example 1, as shown in FIG. 2B. In the case of Comparative Example 1, it can be seen that the silver nanowires were not impregnated onto the nanofibers but intermingled with each other, as shown in FIG. 2E. As a result, it can be seen that some of the pores included in the nanofiber elastic mesh sheet were clogged with the nanowires. To attach the bioelectrode to a part of the human body to use the bioelectrode for a long time, it was very important to secure the air permeability. In the case of Example 1, it was confirmed that the bioelectrode had excellent air permeability and flexibility when a larger amount of the pores was included in the bioelectrode, compared to Comparative Example 1.

Also, when FIG. 2C and FIG. 2F were compared, it was confirmed that the welding point between the silver nanowires was formed in the case of Example 1, whereas the silver nanowires simply come into physical contact with each other without forming a welding point between the silver nanowires in the case of Comparative Example 1.

### (Experimental Example 2) Comparison of Electrical Characteristics

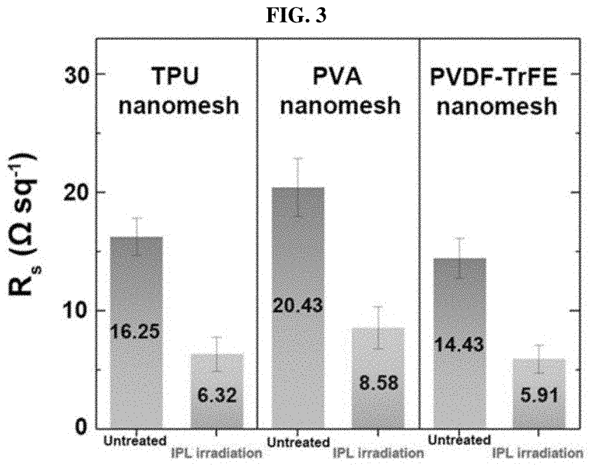

The sheet resistance characteristics of Examples 1 to 3 and Comparative Examples 1 to 3 were compared and analyzed. The results are shown in FIG. 3.

As shown in FIG. 3, it was confirmed that the sheet resistances of Examples 1, 2 and 3 were 6.32 Ω/sq, 8.58 Ω/sq, and 5.91 Ω/sq, which were 2.57-folds, 2.38-folds, and 2.44-folds lower than the sheet resistances of Comparative Example 1 (16.25 Ω/sq), Comparative Example 2 (20.43 Ω/sq), and Comparative Example 3 (14.43 Ω/sq), respectively. As seen from Experimental Example 1, it can be seen that, because the bioelectrode included the welding point formed between the silver nanowires in the case of Examples 1 to 3, the bioelectrode had excellent sheet resistance characteristics due to a significant decrease in contact resistance, compared to Comparative Examples 1 to 3.

In addition, a trend of change in resistance of the fabricated bioelectrode with respect to the periodic strain of stretching and releasing the fabricated bioelectrode was observed.

After a tensile tester was fixed in both ends of the bioelectrode, a stretching cycle includes: stretching a bioelectrode for one second and suspending the bioelectrode for one second, releasing the bioelectrode into an original state, and finally suspending the bioelectrode for one second. This stretching-releasing cycle was performed 500 times.

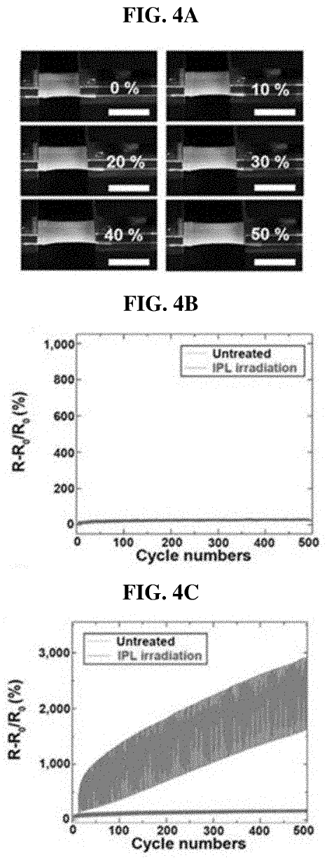

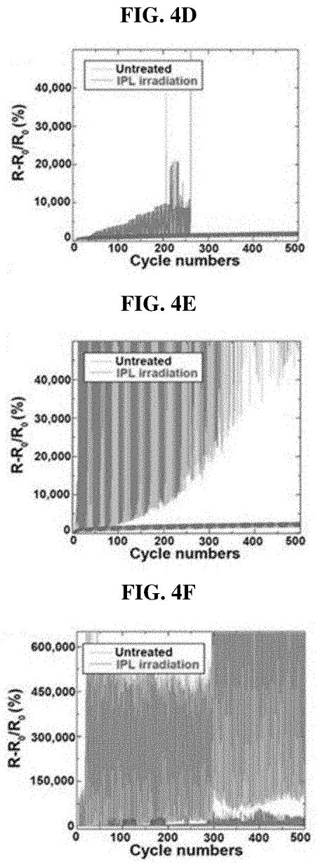

FIG. 4A shows digital images of Example 1 in which the stretchable nano-mesh bioelectrode is stretched by 0%, 10%, 20%, 30%, 40%, and 50%, respectively, and FIG. 4B, FIG. 4C, FIG. 4D, FIG. 4E, and FIG. 4F are diagrams showing the results showing the trends of changes in resistance of Example 1 and Comparative Example 1 when a stretching-releasing cycle is performed 500 times with respect to 10%, 20%, 30%, 40%, and 50% strains, respectively.

As shown in FIG. 4B, it was confirmed that the resistance was not highly varied in both Example 1 and Comparative Example 1 when the stretching-releasing cycle was performed 500 times by applying a strain of 10% to the bioelectrodes of Example 1 and Comparative Example 1.

However, when the stretching-releasing cycle was performed by applying a strain of 20% or more, it was confirmed that a change in resistance was still slight in the case of Example 1, whereas the resistance value was 15-fold higher than the initial resistance value before the strain was applied in the case of Comparative Example 1, as shown in FIG. 4C to FIG. 4E. Based on the results, it can be seen that, when the stretchable nano-mesh bioelectrode of Example 1 had a structure in which the silver nanowire network including the welding point between the silver nanowires was impregnated onto the nanofiber elastic mesh sheet, the stretchable nano-mesh bioelectrode had very excellent durability compared to Comparative Example 1 because the initial electrical characteristics of the stretchable nano-mesh bioelectrode before modification were maintained when the stretchable nano-mesh bioelectrode was subjected to the stretching-releasing cycle and then returned to its original state.

In addition, the stretchable nano-mesh bioelectrode of Example 1 was applied to fabricate a strain sensor, and the performance of the strain sensor was tested.

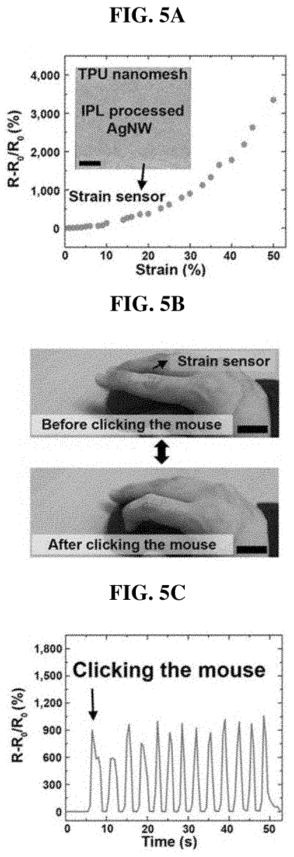

FIG. 5A is a diagram showing a change in resistance according to a degree of strain of a strain sensor, FIG. 5B is a digital image showing situations when a mouse is clicked and when a mouse is not clicked after the strain sensor is attached to a finger, and FIG. 5C is a diagram showing the results of observing a change in resistance in a real-time manner when the mouse is clicked and when the mouse is not clicked in a state in which the strain sensor is attached to a finger.

As seen from FIG. 5A, it can be seen that the strain sensor showed a certain change in resistance according to the degree of strain. In effect, a test was performed after the strain sensor was attached to the skin. As a result, it was confirmed that the change in resistance was clearly detected when the mouse was clicked and was not clicked in a real-time manner, as shown in FIG. 5C.

Hereinabove, although the present invention has been described with reference to the specific subject matters and limited embodiments thereof, the specific subject matters and limited embodiments of the present invention have been provided only for assisting in the entire understanding of the present invention. Therefore, the present invention is not limited to the exemplary embodiments. Various modifications and changes may be made from this description by those skilled in the art to which the present invention pertains.

Therefore, the spirit of the present invention should not be limited to the embodiments as described herein, and the following claims as well as all modifications equal or equivalent to the claims are intended to fall within the scope and spirit of the invention.

1. A stretchable nano-mesh bioelectrode comprising:
a nanofiber elastic mesh sheet comprising polymer nanofibers formed by electrospinning; and
a metal nanowire network having a portion impregnated onto the nanofiber elastic mesh sheet.

2. The stretchable nano-mesh bioelectrode of claim 1, wherein a contact point between metal nanowires in the metal nanowire network comprises a welding point.

3. The stretchable nano-mesh bioelectrode of claim 1, wherein the impregnation is performed so that 20% by volume or more of the metal nanowire network is impregnated in a thickness direction.

4. The stretchable nano-mesh bioelectrode of claim 3, wherein the nano-mesh bioelectrode comprises pores, and the pores are formed by the nanofiber mesh sheet onto which the metal nanowire network is impregnated.

5. The stretchable nano-mesh bioelectrode of claim 1, wherein the polymer comprises one or more selected from an olefin-based elastomer, a styrene-based elastomer, a thermoplastic polyester-based elastomer, a thermoplastic polyurethane-based elastomer, a thermoplastic acrylic elastomer, a thermoplastic vinyl-based polymer, a thermoplastic fluorine-based polymer, and a mixture thereof.

6. The stretchable nano-mesh bioelectrode of claim 5, wherein the polymer has a glass transition temperature of 60° C. or lower.

7. The stretchable nano-mesh bioelectrode of claim 1, wherein a diameter ratio of the metal nanowires and the polymer nanofiber diameter is in a range of 1:5 to 1:100.

8. The stretchable nano-mesh bioelectrode of claim 1, wherein the metal nanowires have a diameter of 1 to 80 nm.

9. The stretchable nano-mesh bioelectrode of claim 8, wherein the metal nanowires have an aspect ratio of 100 to 1,500.

10. The stretchable nano-mesh bioelectrode of claim 1, wherein, when a stretching-releasing cycle is performed 500 times by applying a strain of 20% to the nano-mesh bioelectrode, a change in resistance of the nano-mesh bioelectrode is less than or equal to 5 folds of an initial resistance value before the application of the strain.

11. A strain sensor comprising the stretchable nano-mesh bioelectrode defined in claim 1.

12. A method of fabricating a stretchable nano-mesh bioelectrode, comprising:
a) fabricating a nanofiber elastic mesh sheet comprising polymer nanofibers on a substrate using electrospinning;
b) injecting a metal nanowire ink comprising a dispersion medium onto the nanofiber elastic mesh sheet fabricated on the substrate in the form of droplets using a spray injection method to coat the nanofiber elastic mesh sheet with metal nanowires;
c) sintering the metal nanowires with light to fabricate a metal nanowire network in which some of the metal nanowires are impregnated onto the nanofiber elastic mesh sheet; and
d) removing the substrate.

13. The method of claim 12, wherein, in step b), the metal nanowire ink is injected in the form of droplets in a state in which the substrate is warmed at a temperature of 40 to 130° C.

14. The method of claim 12, wherein the light sintering is performed by irradiation with intense pulsed light (IPL).

15. The method of claim 14, wherein the metal nanowires are irradiated with the light at a light energy of 0.01 to 10 J/cm² for 0.1 to 10 milliseconds (ms).

16. The method of claim 14, wherein a contact area between the metal nanowires is welded by the light sintering in step c) to form a metal nanowire network.
